(19) United States
(12) Patent Application Publication
(10) Pub. No.: US 2012/0022239 A1
(43) Pub. Date: Jan. 26, 2012

(54) PRECIPITATION OF BIOMOLECULES WITH NEGATIVELY CHARGED POLYMERS

(75) Inventors: James Van Alstine, Stockholm (SE); Jamil Shanagar, Uppsala (SE); Rolf Hjorth, Uppsala (SE); Karol Lacki, Uppsala (SE)

(73) Assignee: GE HEALTHCARE BIO-SCIENCES AB, Uppsala (SE)

(21) Appl. No.: 13/144,100

(22) PCT Filed: Jan. 11, 2010
(86) PCT No.: PCT/SE2010/050017

§ 371 Date: Sep. 26, 2011

(30) Foreign Application Priority Data
Jan. 13, 2009 (SE) 0900032-4

(51) Int. Cl. C07K 1/32; C07K 16/00; C07K 14/575

(52) U.S. Cl. 530/388.1; 530/399; 530/389.1; 530/387.1

(57) ABSTRACT

The present invention relates to methods of isolating biomolecules. More particularly, the invention relates to methods for isolating antibodies (mAbs) and related proteins including antibody fragments (Fabs) under conditions where they are positive and relatively hydrophobic and will react with negatively charged polymer to form polymer-protein complexes which precipitate. The isolation can be accomplished using inexpensive and biocompatible negatively charged polymers such as polyacrylic acid or carboxymethyldextran polymers of various molecular weights as precipitant. It occurs at relatively high concentrations of polymer (e.g. 10%) and high salt concentration (>50 mM) and conductivity (e.g. >10 mS/cm) over wide range of pH.

### FIELD OF THE INVENTION

The present invention relates to methods of isolating biomolecules. More particularly, the invention relates to methods for isolating antibodies and other proteins of commercial interest by polymer-protein complex formation and precipitation.

### BACKGROUND OF THE INVENTION

Processing of proteins from various clarified feeds such as milk and plasma routinely occur at very large scales. Production of biopharmaceuticals such as monoclonal antibodies (mAbs) at 100 Kg scale is now routinely discussed by biopharmaceutical producers. This means that even at high mAb titres (10 g per L) fermentation volumes of 10000 liters may need to be processed. Chromatography media with improved capacity, and high flow capability may be suitable to handle such loads using existing large 1.5 to 2 meter diameter columns. However such columns can be difficult to work with, especially if they become fouled with contaminants, and time to process large volumes through such columns will still present scheduling and other challenges. In addition such processes will not be able to handle the possibly even larger volumes associated with future biopharmaceutical production, or processing proteins as food additives, industrial catalysts, etc. It is therefore well appreciated that rapid, cost effective, process volume (and contaminant) reduction operations should be introduced before chromatography or related downstream operations. This is particularly true when dealing with feeds from bacterial, or other complex feed stream sources such as blood, recombinant milk or recombinant plant.

Various novel, cost-effective primary recovery methods are being investigated. Such methods might be used alone or introduced post fermentation to enhance existing target purification processes. Ideally, the methods used should readily interface with existing processes (filter, antiviral and chromatographic), and not lead to undue dilution or contamination of process streams. Methods of interest include aqueous polymer phase partition, and (target or contaminant) flocculation used in conjunction with precipitation or filtration (see references noted below). Target protein flocculation (herein used interchangeably with precipitation) induced by polymers modified with affinity or charged ligands are attractive as they are conceptually similar to the chromatographic approaches commonly used to purify proteins. In addition polymers modified with charged ligands are less expensive than those modified with affinity ligands.

Recent work by others on precipitating clarified feed include use of calcium and phosphate induced flocculation (e.g. US20070066806 A1) or such flocculation aided by uncharged polymers including poly(ethylene glycol) (e.g. WO2008100578 A2), or negatively charged polymers such as polyvinylsulfonic acid (see below). Some also combined contaminant flocculation with filtration (e.g. US20080193981 A1, WO2008079302 A2). In some cases flocculation is able to achieve a degree of selectivity (see US20080193981 A1, also Judy Glynn, BioPharm International, Mar. 2, 2008).

A patent application by Genentech (US20080193981 A1) uses polyvinylsulfonic acid and polyacrylic acid (PAA) for flocculation. The system worked in regimes of lower conductivity (less than 6 mS/cm) and fairly low antibody concentration, appears to have faced challenges redissolving the resulting complexes (i.e. diluting feed streams), and achieved only about 60% reduction in HCP, <90% target mAb recovery, with good (>95%) DNA reduction and maintenance of target protein monomer to aggregate ratios. It is noteworthy that in this work a. (in ref. to [0128] and FIG. 10) “at pH 7 and 1.5 mS/cm complete precipitation was not achieved until PAA had a MW greater than 35,000” and that b. “(in ref. to [0129] and FIG. 33)” at pH 7 and 12 mS/cm complete precipitation (with polystyrene sulfonic acid, PSS) was not achieved until PSS with a MW greater than 220,000 (220 kDa) was used. “The above authors also investigated some positively charged polymers interacting with net negatively target proteins.

Other groups have sought to complex the often net negative (acidic) host cell protein and other contaminants present in recombinant protein feeds and use filtration to isolate such complexed contaminants from target protein (for example, Judy. Glynn, Biopharm International, Mar. 2, 2008; A. Venkiteshwaran, P. Heider, L. Teysseyre, G. Belfort, Selective precipitation-assisted recovery of immunoglobulins from bovine serum using controlled-fouling crossflow membrane microfiltration, Biotechnology and Bioengineering, Published online 6 May 2008 in Wiley InterScience (www.interscience.wiley.com). DOI 10.1002/bit.21964). However such approaches do not offer target concentration (process volume reduction) which can be of prime importance early in a process.

Some limitations related to the above work are best understood if one considers a situation where a solution rich in target protein is titrated by a polymer with the aim to form insoluble protein polymer complexes which are dependent on having more than one polymer interact with more than one protein such that an interconnected complex is formed. As polymer concentration increases relative to target protein concentration one expects a situation where at first the target is in excess (and soluble) to move towards one where target and polymer are at similar concentrations, or otherwise at ratios where complexes may form, and finally a situation where polymer is in such excess that complexes dissociate. In most cases, as is shown in the above Genentech application, low ionic strength may be needed to promote strong protein-polymer interaction. Naturally polymer MW will (as with protein MW) play a complicated role in the complex formation since larger polymer size may favour complex formation in a steric sense but oppose it by reducing polymer molar concentration and diffusion rate.

In regard to the above discussion six points need to be noted. First that complex formation and related phenomena (coacervate phase formation, precipitate formation) are complicated, dynamic processes that involve distribution of polymers, proteins, salts and water between liquid regions enriched in complex (protein-polymer and related water) and noncomplexed components. As such entropic considerations, including protein relative solubility (hydrophobicity) and salt related Hoffmeister series effects (see L. A. Moreira, M. Boström, B. W Ninham, E. C. Biscaia, F. W. Tavares, Hoffmeister effects: Why protein charge, pH titration and protein precipitation depend on the choices of background salt solution, Colloids and Surfaces A, 282-283, (2006) 457-563) as well as partition effects (see H.-O. Johansson, G. Karlström, F. Tjerneld, C. A. Haynes, Driving forces for phase separation and partitioning in aqueous two-phase systems. J. Chromatography B, 711 (1998) 3-17) may play significant roles. Secondly, operations which require low conductivity to generate target capture (i.e. complex formation) mean unwanted dilution of process feed streams which, in larger scale operations, may not be economically feasible. Thirdly operations that require specific conditions of pH and low protein concentration to effect target release (i.e. complex dissolution) may also lead to a need to pH adjust and dilution of process streams. Fourthly specific target release conditions and target concentration when re-dissolved may limit what other downstream separation methods they can be cost effectively coupled with. Fifthly, operations which work optimally with higher MW polymers are more difficult to integrate into processes due to challenges related to increased solution viscosity, removal of soluble contaminating polymer, and greater chance of high MW polymers non-specifically fouling chromatography, filter, pump and other process stream surfaces. Sixthly, care must be taken to understand the nature of the complexes formed with special consideration related to alteration (or protection from alteration) of target proteins if stored for any significant time over hours in the complexes, and ease of release of target protein after proteins are stored in the complex for any time during processing.

Accordingly, there is still a need for a method of isolating antibodies and other biomolecules to high levels of purity, that saves time and cost while being scalable and efficient. In regard to complex formation, flocculation, precipitation or related methods, one can list some desired traits related to these basic unmet needs. The method should work with solutions of relatively high salt concentrations (e.g. 150 mM NaCl) neutral pH and high protein concentrations related to many upstream protein feeds. It should also work with variety of different solutions such as clarified fermentation feed. It should offer good target selectivity and process volume reduction so that it can be used upstream. This includes elimination of contaminants such as (often negatively charged) virus, bacteria, cell debris, toxins and nucleic acid contaminants. It should bind targets such as antibodies or antibody fragments with significant recovery in a manner that allows for good capture and ready release. Release should not require undue dilution or change of pH and should leave the target at a concentration and in a range of solutions which allows ready integration with a variety of other separation methods—especially those common to present processes. It should function even at high protein concentrations and, of course, not involve addition of substances whose removal requires either addition of new unit operations, or significant modification of existing unit operations in order to remove the added contaminants.

For many years biopharmaceutical fermentation, purification and polishing/formulation were often seen as separate process areas. A major reason for this was they typically involved different operations and scales related to large measure to the concentration of target in each area with fermentation at perhaps 1 mg/mL, purification by affinity or ion exchange raising the concentration to perhaps 30 mg/mL with polishing formulation steps taking the target to anywhere from 100 to 200 mg/mL in liquid or solid form. These distinctions are blurring now that antibodies and other biopharmaceuticals can reach 30 mg/mL in fermentation feed, and 100 mg/L or higher in ion exchange chromatography. Formulation often involves combining protein or other biopharmaceutical with excipients such as polymers such as Dextrans™, poly(ethylene glycol)s or Polysorbates™ (polyethoxylated sorbitan and laurate) and various commercially available copolymers or block copolymers of oxyethylene or oxypropylene such as Tergitols™ or Pluronics™. Many excipients can also be charged including use of other proteins (i.e. charged amphipathic biopolymers) such as albumin. In part to stabilize the biopharmaceutical during storage, maintain high concentrations without inducing aggregation, and allowing for rapid dissolving and uptake in the body. Given the above it is natural that any partition or precipitation method which localizes antibodies or other target proteins in solution or insoluble complex with biocompatible polymers should be of interest not only in regard to purification but also formulation and storage of biopharmaceuticals.

### SUMMARY OF THE INVENTION

The present invention relates to methods of isolating biomolecules. More particularly, the invention relates to methods for isolating antibodies (mAbs) and related proteins including antibody fragments (Fabs) under conditions where they are positive and relatively hydrophobic and will react with negatively charged polymer to form polymer-protein complexes which precipitate.

Thus, in one embodiment, the invention provides a method of isolating a biomolecule, comprising the steps of: (a) providing an aqueous sample containing the biomolecule; (b) mixing the aqueous sample with a negatively charged polymer in the presence of a salt, under conditions such that the polymer selectively complex and flocculate the biomolecule to form a mixture of precipitate including the biomolecule; (c) separating the biomolecule precipitate from the aqueous liquid; and (d) resuspending the biomolecule in a resuspension buffer.

The isolation can be accomplished using inexpensive and biocompatible negatively charged polymers such as polyacrylic acid or carboxymethyldextran polymers of various molecular weights as precipitant. It occurs at relatively high concentrations of polymer (e.g. 10%) and high salt concentration (>50 mM) and conductivity (e.g. >10 mS/cm) over wide range of pH (between 5 to 9 depending on various factors). As the method functions in regimes of excess polymer it is not very sensitive to solution protein concentration. Most polymer and salt are not retained in the precipitant and ‘partition’ to the supernatant where they might be recycled. Contaminants such as host cell proteins, nucleic acid (and supposedly other negatively charged contaminants such as virus, bacteria, toxins,) tend to be excluded from the polymer-target protein complex. In various studies 90+ % mAb was recovered in precipitate with 95+ % HCP and DNA recovered in the supernatant.

The process appears to work well with a variety of complex solutions which contain target and other proteins at high concentration (e.g. 10 g/L) and conductivity. These include solutions such as clarified fermentation feed, and target containing phase from aqueous polymer phase partitioning. The precipitates are target protein rich, and readily dissolve at low dilution in variety of aqueous solutions. These solutions may but do not require having low pH. This allows direct integration of the method with a variety of other separation and downstream processing methods and operations.

### BRIEF DESCRIPTION OF THE DRAWINGS

FIG. 1: Polymer protein complex formation and precipitation of Gammanorm human polyclonal antibody (GN) in solutions containing 10% (w/w) NaPAA 8000 at room temperature, and different salt conditions at pH 7.

FIG. 2: Antibody recovery as function of salt conditions in FIG. 1.

FIG. 3: Antibody recovery as function of buffer conductivity (mS/cm) for conditions in FIG. 1. Note that buffer conductivity does not include contribution of the polymer.

FIG. 4: Polymer protein complex formation and precipitation of Gammanorm human polyclonal antibody (GN) in solutions containing 10% (w/w) NaPAA 15000 at room temperature, and different salt conditions at pH 7.

FIG. 5: Antibody recovery as function of salt conditions in FIG. 4.

FIG. 6: Antibody recovery as function of buffer conductivity (mS/cm) for conditions in

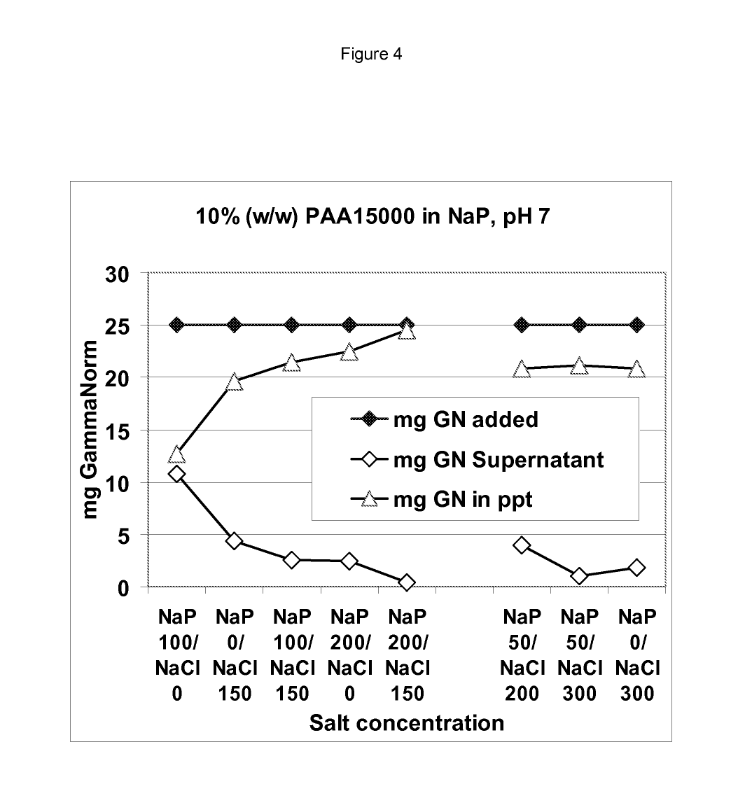

FIG. 4. Note that buffer conductivity does not include contribution of the polymer.

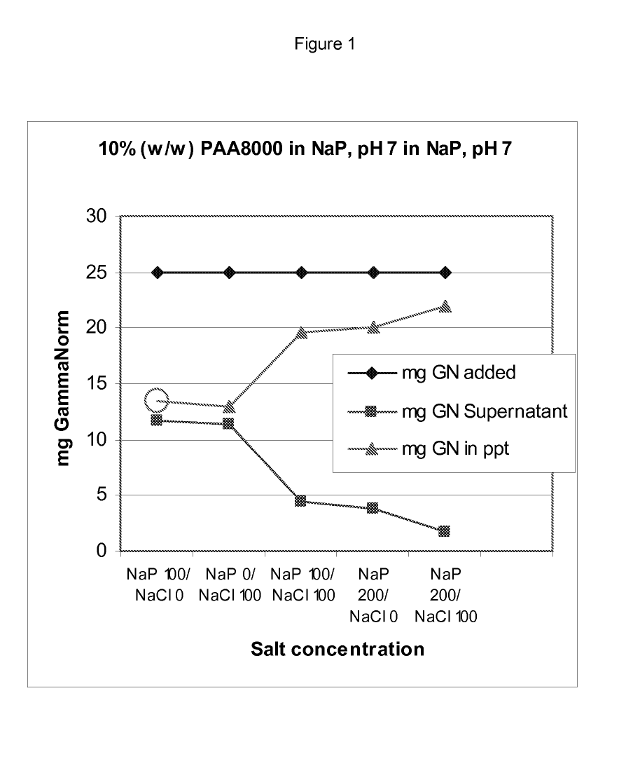

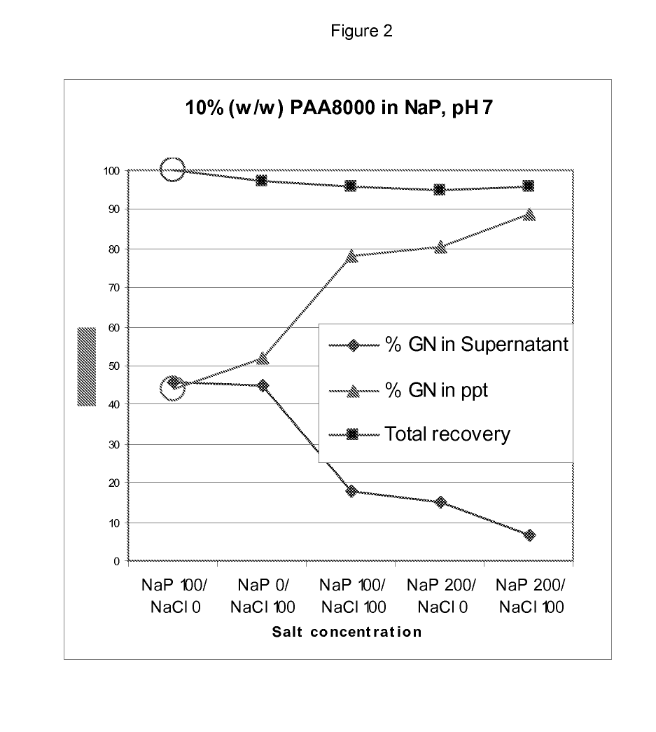

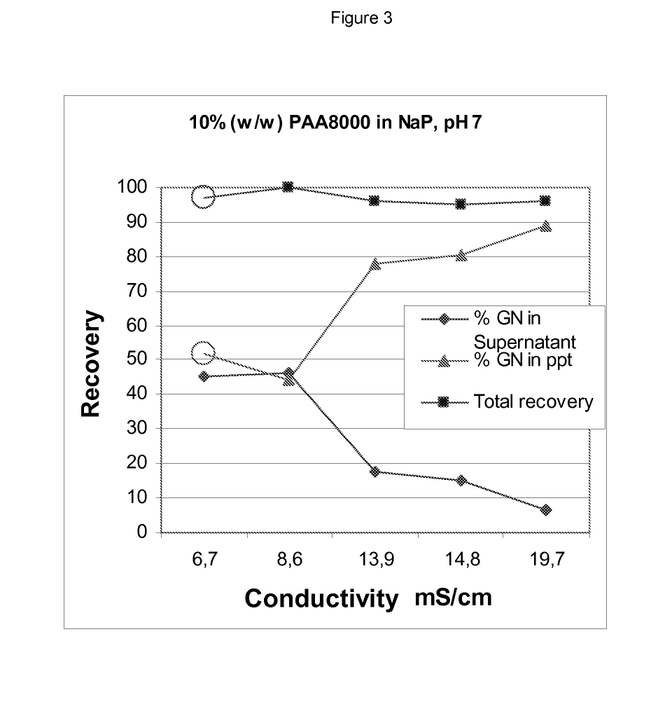

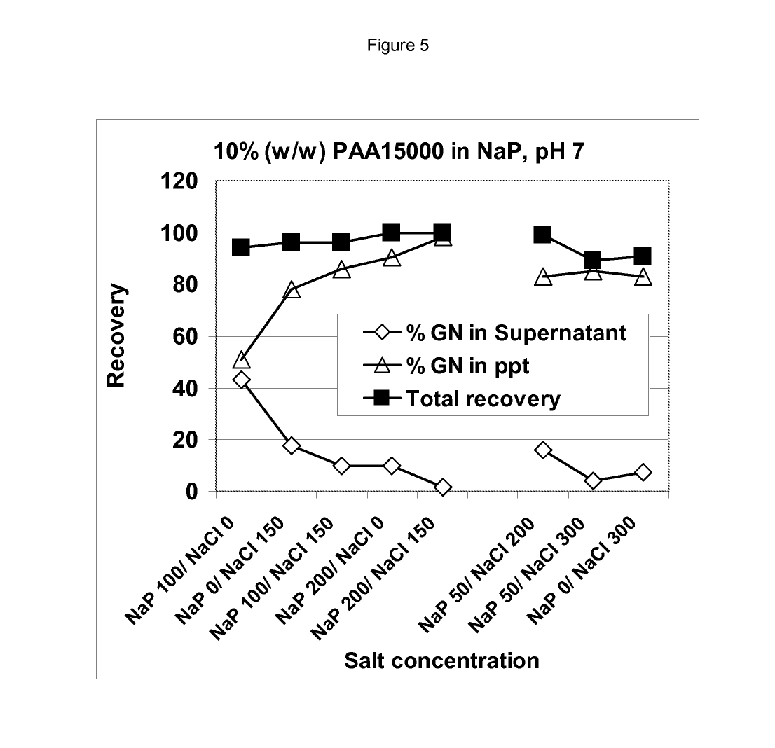

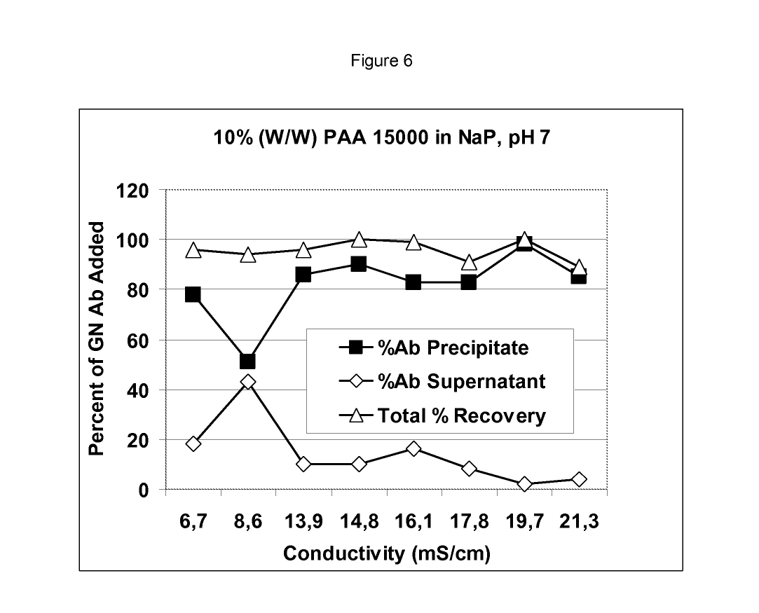

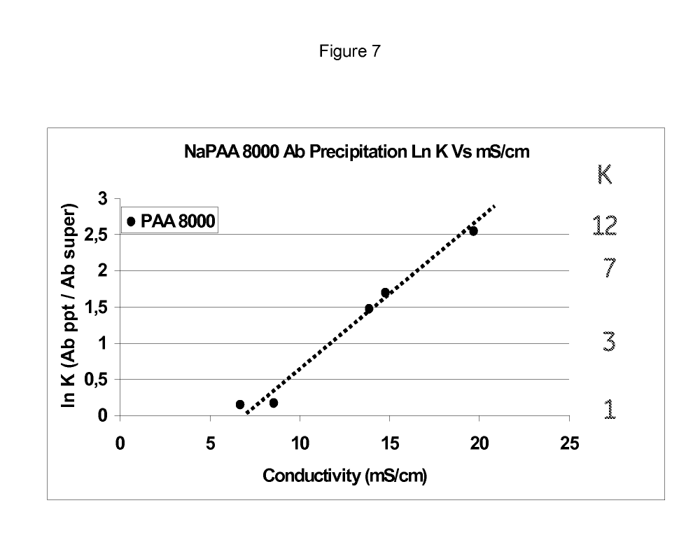

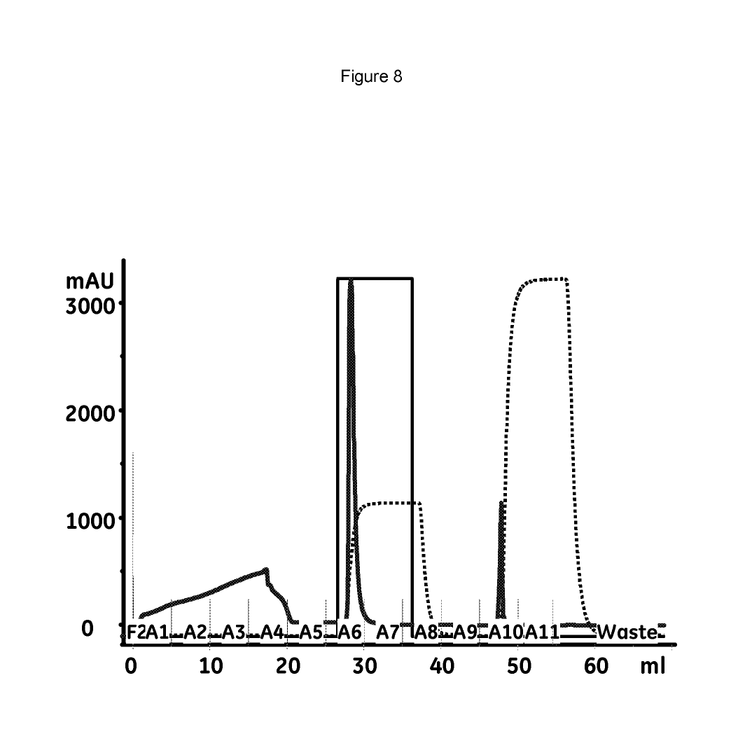

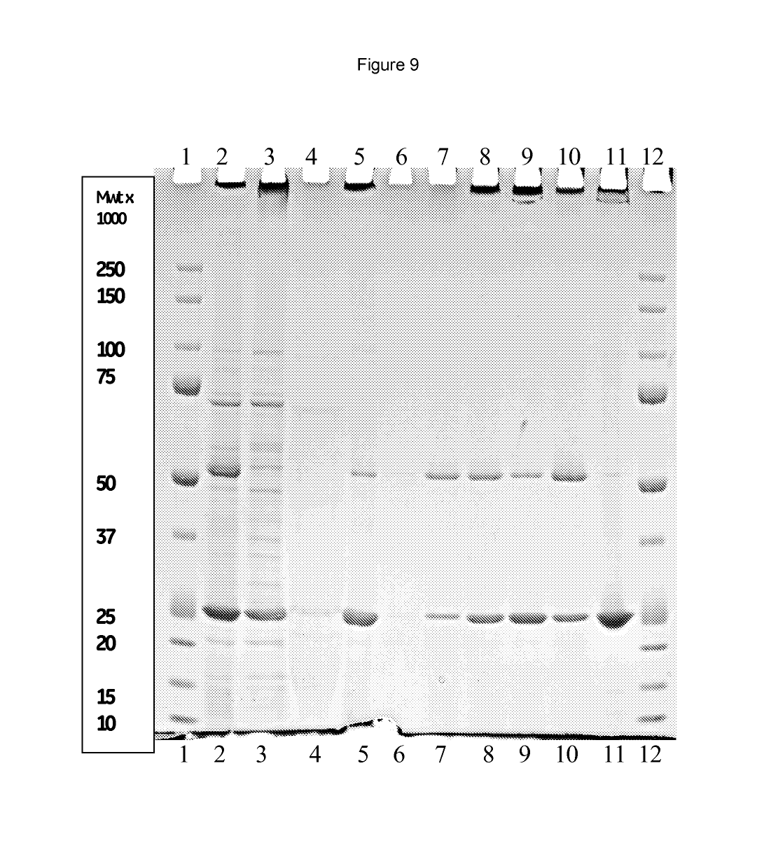

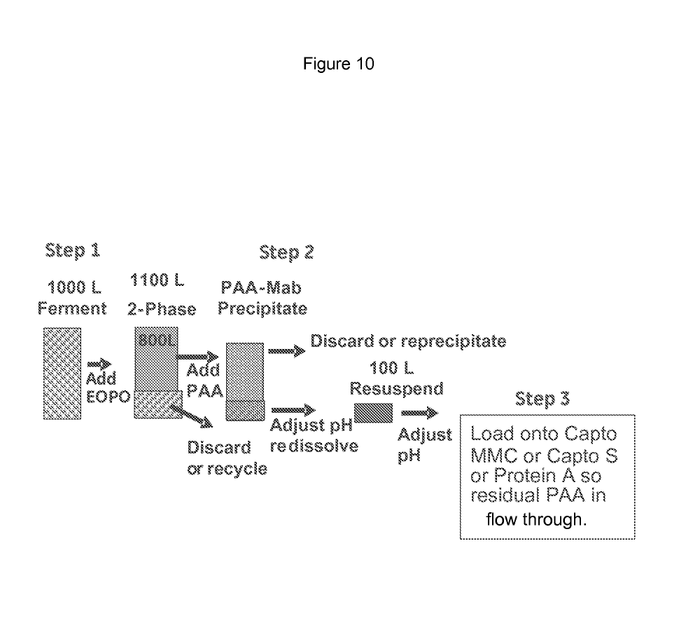

FIG. 7: Plot of K (ratio Ab precipitated/Ab nonprecipitated) and logarithm (Ln) K versus conductivity (mS/cm) for NaPAA 8000 related experiments in FIGS. 1 to 3. Similar direct relationship was also found for results related to FIGS. 4 to 6 (data not shown).

FIG. 8: Chromatography of resuspended mAb precipitate from real Chinese Hampster Ovary (CHO) cell fermentation feed, clarified by aqueous polymer two phase system (APTP) partitioning, on Capto™ MMC multimodal cation exchange chromatoraphy media (GE Healthcare).

FIG. 9: SDS PAGE of applied and collected fractions according to FIG. 8. Lane 1: Molecular weight marker; lane 2: WAVE 51 Feed; lane 3: Wave 51 Feed ATPS; lane 4: Supernatant; lane 5: Resuspended precipitate in pH 5.5; lane 6: Fraction A1; lane 7, Fraction A2; lane 8: Fraction A3; lane 9: Fraction A4; lane 10: Fractions A1-4; lane 11: Fraction A6, eluate; lane 12: Molecular weight marker.

FIG. 10: Outline of three step primary purification scheme for protein based on partition, precipitation and chromatography. In the first step, at least 95% of the protein of interest (mAb) is partitioned to the desired phase. The fermentation broth is also greatly clarified. In the second step, at least 90% of the protein is recovered, with at least 95% reduction of HCP and DNA, as well as significant reduction in the levels of virus and toxin.

### DETAILS DESCRIPTION OF THE INVENTION

In one aspect, the current invention relates to a method for isolating a biomolecule from an aqueous sample containing the biomolecule and impurities. In particular, it is found that negatively charged, carboxy group containing polymers can selectively complex and flocculate (herein termed precipitate) positively charged biomolecules such as antibody from varied aqueous solutions.

The method can be applied to a wide variety of aqueous samples. Such samples include but are not restricted to: fermentation product from a prokaryotic or eukaryotic expression system, blood, recombinant milk, recombinant plant solution, and any other aqueous sample containing the biomolecule of interest. The sample is preferably cell-free and more preferably clarified to remove any solid contaminant. This is achieved by employing any conveniently available method, for example by filtration or by centrifugation. Clarification is also achievable by an aqueous phase partitioning method.

The method is suitable for the concentration and isolation of antibodies. As used herein, the term “antibody” means any recombinant or naturally-occurring intact antibody, e.g. an antibody comprising an antigen-binding variable region as well as a light chain constant domain (CL) and heavy chain constant domains. Also encompassed by the term are antibody fragments, or molecules including antibody fragments, including, but not limited to, Fab, Fab′, F(ab′)₂, Fv and Fc fragments. The term “antibody” specifically encompasses fusion proteins such as Fc fusion proteins, peptibodies and other chimeric antibodies. The term “antibody” specifically encompasses both monoclonal and polyclonal antibodies. In various embodiments the antibody can be an IgG antibody, for example an IgG1, IgG2, IgG3 or IgG4 antibody. Although isolation of antibodies is exemplified below, it is envisaged that the method works equally well for the isolation of many other proteins or biomolecules that can exhibit a net positive change.

The pH of the liquid sample comprising the biomolecule of interest is adjusted to at or below the pl of the target biomolecule or, if more than one target biomolecule such as in case of polyclonal antibody target sample, to at or below the pl of the lowest pl target molecule. The pl of a biomolecule can be readily determined using one of the various methods of determining pl known to those of ordinary skill in the art. In a preferred embodiment, the pl is determined by performing capillary isoelectric focusing (clEF) on a sample comprising the biomolecules and measuring the pl. The adjusting of the pH can be carried out in any convenient fashion, for example by adding aliquots of an acidic solution to the aqueous sample until the pH of the sample falls within the acceptable pH range. It is preferable to achieve and maintain a sample pH between 5 and 9, such as around neutral (pH 7).

Concurrent to or after adjusting the pH of the sample, a negatively charged, carboxyl group containing polymer can be admixed with the sample for selective complexation and flocculation of the biomolecule. Under suitable pH and salt condition, a flocculate is formed containing the biomolecule of interest. Suitably, the polymer is a polycarboxylic acid (PCA) polymer. A person skilled in the art readily understands the principle of choosing the suitable PCA polymer type and degree of substitution (carboxylation) for an effective complex formation. Other polymers would work as well. These include CM cellulose or CM starch as well as polymers that contain monomers similar to acrylic acid. Naturally the polymers may be engineered to exhibit other properties (e.g. size, solubility under certain conditions, magnetism) which further enhance their use with the method. Preferably, highly carboxylated polyacrylic acid (PAA) having a molecular weight greater than 5,000 is used to achieve a selective antibody complexation. Similar result is also obtained with higher MW carboxymethyl-Dextran (CMD) with lower relative degree of carboxylation (degree substitution 1.4). The concentration of the polymer to the aqueous sample is preferably between 3% (w/v) and 30% (w/v), for example 5% (w/v), 10% (w/v) or 15% (w/v).

In a preferred embodiments, the flocculate (precipitate) containing the biomolecules is formed in the presence of a polymer, at around neutral pH, and relatively high conductivity (e.g., >50 mM NaPhosphate). In the most preferred embodiments, the polymer is PAA (MW>5000) or CMD (MW 10000 and 40000, substitution 2.26 and 3.24 mmol/g).

Optionally, one or more salt is present and assists the precipitation process. One of such a salt is sodium phosphate. Typically one must raise conductivity above a threshold where precipitation occurs (e.g. often 5 mS/cm). Some salt specific (e.g. Hoffmeister-type) effects are expected but the operator has some freedom in choice of salt. Alternatively, the salt can be sodium chloride, sodium citrate or sodium sulphate or potassium or other salts or mixture of such salts. Salt and polymer can be added in solid form, which may require additional equilibration time for dissolving and mixing of reagents. Salt and polymer may also be added in form of one or more liquid concentrates, or in some cases in solid form.

The mixture of aqueous sample, polymer and salt is incubated for a period of time between 15 minutes and 24 hours, dependent on added form (see above), mixing (if any) and volume. However typically if liquid concentrates are added with suitable mixing a time of 30 minutes should suffice for most applications. Obviously the time for separation of complex and suspending fluid (supernatant) will depend on the method used to separate them. A few hours for spontaneous complex formation and sedimentation in small volumes (up to test tube size) verus similar time for large volumes subjected to filtration or centrifugation. The length of the incubation can vary with the biomolecule to be isolated and can be optimized by varying the incubation time for a given set of conditions (e.g., polymer concentration, weight, etc.), measuring the amounts of the biomolecule that is precipitated for each incubation period and selecting the incubation period that provides the optimal or desired level of isolation.

Over the course of the incubation period, the mixture can be mixed continuously, at regular intervals, only a desired number of times or not at all. Mixing is not required, but those of skill in the art will recognize when, in the practice of the present invention, mixing may be desirable in the formation of the precipitate.

The incubation can be carried out at any temperature found to be conducive to the formation of the precipitate. For example, the incubation can be performed at a temperature between 2° C. and 8° C. or at room temperature. Fermentation samples are often cooled to room temperature or lower temperature to reduce protease activity during further processing. Indeed, one advantage of the present invention is the ability to perform the incubation step at room temperature, with no need to keep the mixture refrigerated or even set to a particular temperature.

Once flocculate forms, the mixture can be separated into the precipitate and the liquid phase by employing any convenient approach. In one embodiment, the mixture is centrifuged. In this embodiment, the precipitate collects at the bottom of the vessel, while the liquid phase contains most of the impurities. Following centrifugation, the liquid phase is removed, for example by decanting or by aspiration.

In another embodiment, the mixture can be separated by filtration.

Following the separation of the precipitate from the liquid sample, the precipitate can optionally be washed with a buffer. A goal of the optional washing step may be to remove residual liquid component from the precipitate. The optional washing can comprise simply contacting a wash buffer with the precipitate and then removing the wash buffer by aspirating or decanting the buffer away.

The precipitate can be readily resuspended (i.e. re-dissolved) in a aqueous solution including water or buffered solution. This relative freedom of choice in resuspension solution is advantageous in providing for little dilution of target and optimal choice of buffer with little concern other than to maintain target native activity and optimize further separation steps. Preferably, the resuspension buffer is a low ionic strength solution and has a pH of between 4.0 and 9.0. One example of a resuspension buffer is a sodium acetate buffer at pH 5.0.

In the examples below, complex formation with sample solutions containing antibody at <5 g/L typically yields a flocculant <2% volume (and often <1%) of the starting fluid. Therefore precipitation achieves 50 to 100x concentration of the desired biomolecule. Recovery rate for the biomolecule is high (˜90%), so is separation of both host cell proteins and DNA (both ˜95%). The high selectivity may reside in the relatively highly charged, polycationic, large surface area and the relative chain flexibility of the antibody molecules compared to most contaminants—coupled with the greater chain flexibility afforded by polycarboxylate versus polysulfated polymers. As such the complexes may also exhibit reduced levels of virus, toxins and other negatively charged contaminants. There is no specific property of antibodies, such as binding site affinity for specific antigen or ligand, that participates in the separation method. It is thus expected to work for variety of other molecules including antibody fragments.

The significant volume reduction, and ready complex dissolution (resuspension) in a variety of solutions support direct integration of the current flocculation method with standard downstream purification processes. Thus, following resuspension of the precipitate, the resuspended biomolecules in solution can be further processed by one or more additional purification steps such as chromatography, in either flow-through or capture mode for the biomolecule or residual polymer, so as to further purify the desired biomolecule.

Thus in one embodiment, the resuspended biomolecules are captured on an affinity media (e.g., protein A media). The target biomolecule is then eluted and subjected to polishing by possibly a cation exchange (target capture) step, or a mixed mode (target flow through, contaminant capture) step.

In an alternative embodiment, the target biomolecule is loaded onto cation exchange media where the polymer flows through. In a different embodiment, the biomolecules is resuspended in higher ionic strength solution, and is directly loaded onto a hydrophobic interaction chromatography column, a mixed mode or affinity column.

As described above, the residue polymers in the precipitate can be removed by scavenging using a capture chromatography media. Alternatively, the polymers can be removed by other methods, such as by phase partition of an aqueous multiphase system.

In addition, the residual polymers in the precipitate could be removed by allowing them to flow through a chromatographic or filtration or other (monolithic) capture media which significantly adsorb (capture) the target but not the polymer. Alternatively, the residual polymers in the precipitate can be removed by allowing it to flow through a chromatographic or filtration or other (monolithic) size exclusion media which has different rate of flow or degree of hindrance for the polymers than the target.

Thus, further provided is a method of using Capto MMC and related capture media designed for use with high conductivity solutions to purify target containing solution produced by the above methods.

It is noted that all embodiments of the present invention can be employed on any scale. For example, the present invention can be applied to large scale biomolecule production operations in which biomolecules are isolated from tens, hundreds or thousands of liters of cell culture media. In another example, the present invention can be employed on a smaller scale, for example in bench-top scale operations in which biomolecules are isolated from volumes on the order of several liters of media or even volumes of much less than a liter of media.

Containers used for the novel methods may be fixed or disposable and may be modified in various obvious ways to enhance target recovery and removal of liquid supernatant. So too as the method involves only addition of polymer and salt solution to the target containing feed it can easily be run in on line or continuous processing modes (e.g. using filtration rather than sedimentation or centrifugation to isolate complex). As a liquid method which is not dependent on a solid support it is readily possible to employ high throughput screening (e.g. at milliliter scale volumes in microtitre plates) to optimize various parameters such as target recovery and contaminant removal under conditions which allow for use of minimal expensive target protein and feed.

The simplicity and robust nature of the method suggests a number of other exciting possibilities. For example the pH dependence suggests that it might be run using partial CO2 pressure to vary pH in a carbonate buffered solution so as to move back and forth between pH conditions where complex will form or dissolve. The post precipitation redissolving step might be combined with lowering pH to effect killing of residual virus. Another possibility is to combine the precipitation with aqueous polymer two phase partitioning in polymer-salt, polymer-polymer or thermoresponsive (reverse thermo solubility) polymer two phase systems. In the latter systems polymer will self associate at a cloud point temperature (Tc) and form a water and (target) protein rich phase floating on a polymer rich phase. Partition in such systems is capable of affecting a rapid initial clarification (removal of cells and cell debris) of feed at unit gravity i.e. without use of centrifugation (Swedish patent application 0900014-2, by James Van Alstine, Jamil Shanagar and Rolf Hjorth, filed on Jan. 8, 2009, entitled: “Separation method using single polymer phase systems”, the disclosure of which is hereby incorporated by reference in its entirety) and some removal of contaminants. And it does so at conductivities associated with recombinant or other large scale protein rich feeds (including those associated with plasma, blood or recombinant plant target containing feed streams). However it does not provide much target concentration or HCP removal. As such it is ideal for combining with the present method so as to generate a two step clarification and purification regime easily inserted into existing or new processes (Table 1). This includes processes designed to work entirely with disposable components. Various other obvious possibilities exist, such as carrying out the operation in containers where target and contaminants are free to move between two compartments but the complexing polymer is retained by being localized by terminal covalent bonding, or on basis of MW by a filter which allows target to pass but not a polymer of much higher MW than target.

TABLE 1
Main Operational Attributes of Aqueous Polymer Phase Systems and New Polymer
Precipitation Method Employed Alone or Following a Partition Operation.

| | | Classic 2 | 1 | Thermo | | |
|---|---|---|---|---|---|---|
| | | Polymer | Polymer + | Responsive | New | Thermo |
| | | Phase | Hi Salt | Polymer | Pptn | System + |
| | Property | Systems | Systems | Systems | Method | Ppt'n |
| 1 | Technically simple and robust operation. | + | + | + | + | + |
| 2 | Readily integrate target containing | + | − | + | + | + |
| | phase with follow on operations and | | | | | |
| | processes. | | | | | |
| 3 | Fast (kiloL/hr) processing. Handles | − | − | + | + | + |
| | future loads (>KL, >20% solids, >10 | | | | | |
| | g/L target expression) | | | | | |
| 4 | Affordable (<<10 Euro/L/step) without | − | − | + | + | + |
| | recycling of polymers and salts. Process | | | | | |
| | Savings > Estimated Cost. | | | | | |
| 5 | Target (e.g IgG) recovery >90% with | + | ? | + | + | + |
| | little expected denaturation or alteration. | | | | | |
| 6 | Effect 1° clarification w/o centrifugation. | + | + | + | − | + |
| 7 | Target partition into phase which | − | − | + | + | + |
| | contains little (e.g 1%) residual polymer. | | | | | |
| 8 | Significant HCP, DNA, and possibly | − | − | − | + | + |
| | other contaminant (e.g. virus) removal. | | | | | |
| 9 | Inexpensive, nontoxic, reagents. | − | − | + | + | + |
| | Removed without extra unit operations. | | | | | |
| 10 | Easily validated operation/process. | + | + | + | + | + |
| 11 | Able to be optimized via HTPD, with | + | + | + | + | + |
| | ease of scaling and ready modelling, | | | | | |
| 12 | Suits varied fermentations, e.g. | + | ? | + | + | + |
| | eukaryotic CHO or bacterial E. coli cells. | | | | | |

+ = affirmative, − = negative, ? = varied or unknown, HTPD = high throughput process development, HCP = host cell protein, KL = 1000 liter

FIG. 10 takes some of the above concepts, and based on experimental details noted below, outlines a three step primary purification scheme for protein based on partition, precipitation and chromatography. Entire process can be run using disposable components. In step 1 fermentation sample or recombinant cell (rCell) or rBacteria (or plant or animal related target containing solution) is subjected to aqueous polymer phase partition to clarify solution of cell and other particulate debris. The target containing phase (in the example the water rich phase from thermoseparated ethylene oxide propylene oxide or EOPO type polymer based one polymer two phase system) is isolated and then adjusted via addition of salts and pH and protein complexing polymer (in the example PAA for a net positively charged mAb protein it readily complexes with). Complex formation and isolation of complex (by sedimentation, centrifugation or filtration) follows. At this step the supernatant can be subjected to another round of precipitation to enhance recovery of target in precipitate. The complex can then be re-dissolved in fresh buffer. This might be a low pH (e.g. pH 4) buffer as part of antiviral treatment. In the third separation step the solution containing the re-dissolved target protein is subjected to affinity, mixed mode, ion exchange, or other separation based method such as capture chromatography. It might also be processed by size exclusion or other methods related to filtration, chromatography, monolith columns, etc.

### EXAMPLES

The present examples are provided for illustrative purposes only, and should not in any way be construed as limiting the invention as defined by the appended claims.

### Materials and Investigated Units:

### Chemicals:

GammaNorm (human polyclonal IgG): 165 mg/ml, pl approx. 7, Octapharma.
Polyacrylic acid Na-salt (PAA): 35% (w/w), MW=15 000; 45% (w/w), MW=8 000; MW=5100 and MW=2100, all from Aldrich.
Carboxymethyldextran (CMD) 0.39 (2.26 mmol/g) MW 40,000 from Meito Sangyo, Japan.
Carboxymethyldextran (CMD) 1.39 (3.24 mmol/g) MW 40,000 from Meito Sangyo, Japan.
Dextran Sulfate (16 to 19% substitution) MW 10,000, 40,000, 100,000 and 500,000) from P K Chemicals, Denmark.
Capto™ Q (17-5316-10) and HiTrap Capto MMC column (11-0032-73) are from GE Healthcare, Uppsala, Sweden.

All other chemicals used in this study were of analytical grade and purchased from MERCK.

### Thermoresponsive Polymer for Phase System Formulation

Breox 50 A 1000 (equal copolymer ethylene oxide and propylene oxide (EOPO): Mw 3 900 See below.

Unless noted EOPO polymer refers to Breox 50 A 1000 which is a random copolymer consisting of 50% ethylene oxide and 50% propylene oxide with a molecular mass (number average) of 3900 Daltons. It is FDA approved for some applications and was obtained from International Specialty Chemicals (Southampton, UK) which is now part of Cognis (www.cognis.com)

### Phase Systems

In small scale studies two phase system (ATPS) solution was prepared directly in a 10 ml Sarstedt tube (unless otherwise stated) by mixing appropriate amounts/volumes of the stock solutions listed below. The final volume of each system was typically 5 ml. The mixtures were vortexed about 30 seconds and were then left for phase formation for about 15 min at 40° C in a water bath.

### Stock Solutions:

EOPO, 20% (w/w): Prepared by dissolving 10 g EOPO in 40 g MQ water.
EOPO, 40% (w/w): Prepared by dissolving 20 g EOPO in 30 g MQ water.
NaP (Na-phospahte, 0.8M): Different pHs (pH 5, 6, 7, 8) were made by mixing 0.8 M
   NaH₂PO₄ and 0.8 M Na₂HPO₄
NaCitrate (0.8 M): A stock solution of pH 7 was prepared by mixing 0.8 M Na₃Citate
   and 0.8 M Citric acid
NaCl (5 M): Prepared by dissolving 14.6 g NaCl in 50 ml MQ water.

### Real Feed Samples:

The real feed mAbs P4 and P5 and Wave 51 were obtained internally from GE Healthcare, Uppsala, Sweden. They were Chinese Hampster Ovary (CHO) cell based fermentations.

### Precipitation Experiments

### Stock Solutions:

NaP (Na-phosphate, 0.8 M, pH 7): prepared by mixing 0.8 M NaH₂PO₄ and 0.8M Na₂HPO₄
NaCl (5 M) Prepared by dissolving 146 g NaCl in 500 ml MilliQ water.

### Methods:

Preparation of polymer solutions: Polymer and salt/buffer solutions for precipitation experiments were prepared by mixing appropriate amounts/volumes of the polymers with appropriate amounts/volumes of the stock solutions listed. Unless noted polymer densities were assumed to be 1.

The required amount of polymer and salt/buffer solutions (Table 2) was mixed and antibody was added to it. The mixture was then kept at room temperature for about three hours (to complex and flocculate) and then was centrifuged at 3000×g for 15 minutes. The supernatant was then isolated from the precipitate. The separated precipitate was resuspended in water or appropriate buffer solution.

Electrophoresis: Run on Phast System (GE Healthcare, Uppsala) under normal operating conditions as gradient of 4 to 12% polyacrylamide and sodium dodecyl sulphate (SDS) reducing gel with 150 V, 1 h, with 10 min staining using Coomassie Blue®.

Chromatography was run according to the chromatography media supplier's recommendations—available from GE Healthcare, Uppsala, Sweden.

### Analytical Chromatography

Protein A Affinity Chromatography Determination of mAb: The selectivity of protein

A interaction for antibody capture allows it to be used for analytical purposes so as to bind all the antibody in a sample while letting 90+ % of contaminants pass the column. Concentration of mAb was measured using a MabSelectSure column. 50 μl samples were applied to a 1 ml HiTrap MabSelectSure column. The area of the eluate peak was integrated and multiplied with the feed and water phase volume respectively. The recovery for the extraction using the ATPS was calculated by comparing the total number of area units. The recovery of mAb for the MabSelectSure step was calculated in the same way. Sample: 50 μl feed or water phase, Column: 1 ml HiTrap MabSelectSure, Buffer A: PBS, Buffer B: 100 mM sodium citrate pH=3.0, Flow 1 ml/min (150 cm/h) Gradient: 0-100% B, step.

Size Exclusion Determination of Aggregate Levels: Dimer and aggregate (and also the mAb concentration) was measured using a Superdex 200 5/150 GL gel filtration (size exclusion chromatography or SEC) column. The area of the dimer-and monomer peak were integrated automatically by the UNICORN software. The total area of the dimer from the feed and the water phase was compared. Sample: 50 μl feed or water phase, Column: 3 ml Superdex 200 5/150 GL, Buffer: PBS, Flow 0.3 ml/min (45 cm/h).

### Larger Scale Fermentation and Partition Based Clarification in Disposable Wave Bioreactor

The real feed mAb cell culture is expressed in 51 CHO cell line (supplied internally). Culture duration was 18 days and culture vessel WAVE Bioreactor system 20/50 with a 20L bag and pH/Oxywell. Culture media was PowerCHO2 (Lonza) with 5 g/L hydrolysate UF8804 (Millipore) and supplied with glucose and glutamine when needed. Feed sample was defined as ready to harvest when the average viability of cells fell below 40%. The contents of the WAVE bag was temperature stabilised at 42° C. when polymer-salt solution was added.

An aqueous polymer two phase system was prepared directly by pumping the stock solution mixture into the WAVE bag which contained 9.5 kg mAb feed. This was 3.6 L of 50% Breox EOPO polymer stock solution, 4.5 L of 800 mM NaPhosphate (NaP) pH 8 and 0.27 L of 5M NaCl stock solution so that total of 8.37 L was added to 9.5 kg of feed. This gave approximate final concentration of 10% EOPO (w/w), 200 mM NaPhosphate, pH 8.0, and 150 mM NaCl. Added stocks were at 40° C. which allowed for feed and phase system mixture to rapidly equilibrate at 40° C. The time for pumping the polymer mixture was about 50 min. After leaving the mixture for shaking on the WAVE reactor for about 15 min the WAVE bag including the bag holder was disconnected from the reactor and was then put on a lab bench with long axis in vertical position. This aided visualization of phase formation but also allowed bag tubing port to be directed to the bottom and top of the bag. It also adjusted the phase height more in keeping with what might be expected in an even larger process (see discussion above). The formation of two phase system was observed after 5 min and was completed after 30 min. A layer of cell debris was formed at the interface. The upper phase was then transferred into different bottles by inserting a tube from the upper part of the WAVE bag which was then connected to a peristaltic pump. The bottom (polymer) phase was then transferred into bottles using a tube attached to what becomes the bottom corner of the WAVE bag when it is placed long axis vertical.

The collected upper phase materials from different bottles were pooled (˜13.5 L) and were then filtrated using a 6 inch ULTA 0.6 μm GF connected to a 6 inch ULTA HC 0.2 μm filter. After filtering of 7 liter material the 6 inch ULTA 0.6 μm GF was replaced with a new filter because of increase of the pressure to 2.5 bar. The filtered material was collected in a WAVE bag and was then kept at 4° C.

The recovery of the mAb in the upper polymer poor phase fractions after ATPS was measured using MabSelect Sure analysis (see above). The mAb recovery and host cell protein (HCP) data for crude feed and the recovered mAb after ATPS experiments were also analysed. The results from these experiments showed that mAb was partially purified by partition with a recovery of >99% with significant removal of cell debris. No significant reduction of HCP was obtained with this system which was chosen to optimize antibody recovery.

### Other Assays

Host Cell Protein (HCP) assay was by commercial enzyme linked immunoassay (Gyrolab). DNA was analysed by standard commercial PicoGreen dye DNA analysis method.

### Example 1

### Screening of Polymers for Antibody Precipitation

Preliminary research (Table 2) suggested that addition of approximately 10% (w/w) deprotonated polyacrylic acid, i.e. sodium polyacrylate (noted below interchangeably as NaPAA or PAA) to suitably buffered human polyclonal antibody solutions results in precipitation of a broad range of human antibodies represented by Gammanorm polyclonal Ig (Octapharma) sample. Such effects can also be reproduced using higher concentration (20% w/w) of higher MW carboxymethyl modified dextrans (CM dextrans, Meito Sangyo, Japan). It is noteworthy that ≧50 mM NaP buffer appears necessary to initiate flocculation. This buffer concentration is similar to the salt concentration needed for NaPAA to form two phase systems with polyethylene glycol (PEG) and appears related to need for buffered control of pH to offset influence of polymer based acid groups—in addition to any need for NaCl or NaP salt to provide entropic driving force for phase formation. (for discussion see H. O. Johansson et al, 1998 and L. A. Moreira et al. 2006).

TABLE 2
Antibody precipitation using different polymers and conditions

| Polymer, (MW kDa) and % (w/w) | | Salts (M) and pH | IgG mg/ml¹ | | Precipitation |
|---|---|---|---|---|---|
| Dextran Sulfate | (500), 4% | 0.1M NaP, pH 7 | 6 | mg/ml | No |
| | (500), 10% | 0.06M NaP, pH 7 | 4 | mg/ml | No |
| | (100), 10% | 0.2M NaP, 0.15M | 6 | mg/ml | No |
| | | NaCl, pH 7 | | | |
| | (10), 10% | 0.2M NaP, 0.15M | 6 | mg/ml | No |
| | | NaCl, pH 7 | | | |
| | (10), 10% | 0.2M NaP, 0.15M | 16 | mg/ml | No |
| | | NaCl, pH 7 | | | |
| | (10), 20% | 0.2M NaP, 0.15M | 6 | mg/ml | No |
| | | NaCl, pH 7 | | | |
| | (10), 10% | 0.1M NaP, pH 7 | 6 | mg/ml | No |
| CM Dextran² | 0.39 sub, (40), 4% | 0.2M NaP, 0.15M | 6 | mg/ml | No |
| | | NaCl, pH 7 | | | |
| | 1.39 sub, (40), 4% | | 6 | mg/ml | No |
| | 1.39 sub, (40), 20% | | 4.5 | mg/ml | Yes |
| NaPAA | (8), 9% | 0.2M NaP 0.15M | 2.5 | mg/ml | Yes |
| | | NaCl, pH 7 | | | |
| | (8), 9% | 0.1M NaP, pH 7 | 6 | mg/ml | Yes |
| | (8), 9% | Water pH ~ 7 | 6 | mg/ml | No |
| | (8), 9% | 0.1M NaP, pH 5 or 4 | 6 | mg/ml | No |

¹IgG = Gammanorm ™ polyclonal human IgG from plasma (Octapharma).
²NormCM dextran MW 40000 and degree of substitution 0.39 or 1.39 (2.26 or 3.24 mmol/g).

The conditions for preliminary screening are given in Table 2. Under these conditions approximately 1 gram of NaPAA containing solution was added to 4 ml buffer containing solution with approximately 75 microliters or more of Gammanorm polyclonal human antibody (165 mg/ml) so as to achieve antibody concentration of 2.5 to 16 mg/ml (g/L) in 5 ml total system volume. It was not possible to induce Ab flocculation with sulphated dextran even when MW of dextran raised to 100000 and the concentration raised to 20%. However in the above studies precipitation occurred with NaPAA (MW 8000) from less than one to over 30 mS/cm solution and mAb concentrations even at 6 g/L or higher with final concentrations of 200 mM NaPhosphate pH 7 and 150 mM NaCl, with much higher polymer concentration. Resuspeding the precipitate (which typically appeared to represent less than 2% of original sample volume in these studies) in ≦1 ml of 100 mM NaP, pH 5 caused dissolution of the complex with an approximately 10 to 20 fold reduction in sample volume (i.e. 10× to 20× fold concentration). It should be noted that assuming protein and polymer density of approximately 1 even an antibody concentration of 16 g/L Ab would represent less than 2% of the sample volume. If (to speculate further) antibody was combined with equal mass of polymer and bound water the resulting complex would still represent <6% of the total system and thus afford a >16 fold concentration in antibody.

### Example 2

### Effect of PAA Polymer MW, Buffer and Salt Concentration on Antibody Precipitation

Gammanorm (GN) human polyclonal IgG antibody (Ab) was used for further precipitation studies. A polyclonal antibody sample was used to ensure that results related to the majority of antibody present would relate to a broad range of antibodies, not just one particular monoclonal antibody. Different molecular weights of PAA (NaPAA) were combined with varied buffer and salt concentrations (see Table 3). The final concentration of PAA was 10% (w/w) and the volume of each system was 5 ml. The result showed that under these conditions little precipitation was achieved with PAA polymers with molecular weights up to 5000. Such precipitation might be possible if polymer or salt concentration is increased. Precipitation of antibody was obtained when the molecular weight was raised to 8000 or 15000 and at high buffer or NaCl salt concentrations. However, minor precipitation was observed when buffer and NaCl were excluded from the systems. This indicates that relatively high buffer concentration or high conductivity is required for precipitation of the antibody with PAA polymers.

TABLE 3
Antibody precipitation using different MW of PAA polymer and
conditions

| NaPAA | Polymer | | pH 7 [NaP] | [NaCl] | Significant |
|---|---|---|---|---|---|
| MW | (w/w) % | Ab (mg/ml)* | (mM) | (mM) | Precipitate. |
| 2100 | 10 | 5 | 100 | 150 | − |
| 5100 | 10 | 5 | 100 | 150 | Very Little |
| 8000 | 10 | 5 | — | — | + |
| 8000 | 10 | 5 | — | 150 | +++ |
| 8000 | 10 | 5 | 100 | | +++ |
| 15000 | 10 | 5 | | | + |
| 15000 | 10 | 5 | 100 | | +++ |
| 15000 | 10 | 5 | | 150 | +++ |
| 15000 | 0 | 5 | | 150 | − |
| 15000 | 10 | 5 | 50 | 200 | +++ |
| 15000 | 10 | 5 | 50 | 300 | +++ |
| 15000 | 10 | 5 | 0 | 300 | +++ |

*150 microliters of Gammanorm concentrate (165 mg/ml) added to 5 ml total is approximately 5 g/L final.
Note
− = none,
+ = some,
+++ = extensive complex related precipitate formation.

### Example 3

### Effect of PAA MW and Salt Concentration on Antibody Precipitation and Recovery

To investigate the effect of buffer and salt concentration on precipitation and antibody recovery, 5 ml total systems with 5 g/L antibody were prepared using NaPAA of 8000 or 15000 MW at different buffer and salt concentrations (Tables 4 and 5). After removing the supernatant from each tube the precipitate was resuspended in 1 ml water and absorbance of each was monitored spectrophotometrically at 280 nm. This allowed calculation of the amount of antibody recovered in the precipitant and supernatant. The results in Tables 4 and 5 and FIGS. 1 to 7 indicate that complexation and recovery of antibody increases with buffer salt concentration (i.e., conductivity). Even under these relatively small scales and manual operation methods recoveries of 80 to 90% were possible to achieve at buffer/salt total concentrations ≧200 mM. Total recovery was typically >95% suggesting that even at 100 mM NaP, 100 mM NaCl it would be possible to effect a double precipitation and secure >90% mAb in precipitate.

TABLE 4
Precipitation and recovery of antibody using 10% PAA 8000.

| | | Ab added | mg Ab | mg Ab | % Ab in | % Ab | Total Ab |
|---|---|---|---|---|---|---|---|
| Expt. | mM NaP/mM NaCl | (mg) | Supernatant | in ppt | Supernatant | in ppt | recovery |
| 1 | NaP 100/NaCl 0 | 25 | 11.62 | 13.4* | 46 | 54* | 100* |
| 2 | NaP 0/NaCl 100 | 25 | 11.36 | 13 | 45 | 52 | 97 |
| 3 | NaP 100/NaCl 100 | 25 | 4.46 | 19.6 | 18 | 78 | 96 |
| 7 | NaP 200/NaCl 0 | 25 | 3.75 | 20.1 | 15 | 81 | 96 |
| 8 | NaP 200/NaCl 100 | 25 | 1.69 | 22 | 7 | 89 | 96 |

*Precipitant re-dissolved in 100 mM NaP pH4.5. Recalculated values (not from AU determinations).
Total recovery refers to Ab in precipitate and supernatant.

TABLE 5
Precipitation and recovery of antibody using 10% PAA 15000.

| | | Ab added | mg Ab | mg Ab | % Ab in | % Ab | Total Ab |
|---|---|---|---|---|---|---|---|
| Expt. | mM NaP/mM NaCl | (mg) | Supernatant | in ppt | Supernatant | in ppt | recovery |
| 4 | NaP 100/NaCl 0 | 25 | 10.76 | 12.7 | 43 | 51 | 94 |
| 5 | NaP 0/NaCl 100 | 25 | 4.42 | 19.6 | 18 | 78 | 96 |
| 6 | NaP 100/NaCl 100 | 25 | 2.51 | 21.5 | 10 | 86 | 96 |
| 9 | NaP 200/NaCl 0 | 25 | 2.44 | 22.5* | 10 | 90* | 100* |
| 10 | NaP 200/NaCl 100 | 25 | 0.45 | 24.5* | 2 | 98* | 100* |
| 11 | NaP 50/NaCl 200 | 25 | 4.01 | 20.8 | 16 | 83 | 99 |
| 12 | NaP 50/NaCl 300 | 25 | 1.05 | 21.2 | 4 | 85 | 89 |
| 13 | NaP 0/NaCl 300 | 25 | 1.87 | 20.8 | 8 | 83 | 91 |

*Precipitate re-dissolved in 100 mM NaP pH 4.5. Recalculated values (not from AU determinations)

### Example 4

### Antibody Precipitation Using Carboxymethyldextran (CMD)

This study was done to verify that other polymers were capable to performing in manner similar to polyacrylic acid. Test polymer was quite different than PAA as it was not used in sodium form and was a carboxymethyl group (CM) modified polysaccharide dextran (D) from a natural bacterial source and not (as in case of PAA) a synthetic polymer where the acidic groups are part of the monomeric structure. Two CMD polymers with different molecular weights (10000 and 40000) were tested for precipitation of polyclonal IgG Gammanorm at concentration of 20% (w/w) in solution of 150 mM NaCl, and 200 mM NaP, pH 7. The result showed that antibody can be precipitated under these conditions. However, no precipitation was obtained when NaP buffer and NaCl were excluded. (Table 6).

TABLE 6
Ab precipitation with 20% w/w CMD of different molecular weight.

| | Carboxyl | CMD | [Carboxyl] | Ab | pH 7 [NaP] | [NaCl] | Ppt. |
|---|---|---|---|---|---|---|---|
| CMD MW | (mmol/g) | (w/w) % | (M) | (g/ml) | (mM) | mM | Formation. |
| 10000 | CMD L-0.91 | 20 | 0.53 | 5 | 0 | 0 | none |
| | (2.66) | | | | control | control | |
| 10000 | CMD L-0.53 | 20 | 0.33 | 5 | 200 | 150 | extensive |
| | (1.65) | | | | | | |
| 10000 | CMD L-0.91 | 20 | 0.53 | 5 | 200 | 150 | extensive |
| | (2.66) | | | | | | |
| 40000 | CMD 0.93 | 20 | 0.45 | 5 | 200 | 150 | extensive |
| | (2.26) | | | | | | |
| 40000 | CMD 1.39 | 20 | 0.64 | 5 | 200 | 150 | extensive |
| | (3.24) | | | | | | |

### Example 5

### Effect of CMD MW and Ligand Density on Antibody Precipitation and Recovery

To investigate the effect of the molecular weight of CMD polymer on precipitate formation and the recovery of Gammanorm antibody in different systems, CMD polymers of two different MW (10000 and 40000) and grafted CM ligand densities were studied at 20% (w/w) in 1.2 ml systems with 150 mM NaCl, 200 mM NaP, pH 7 (Table 7). After removing the supernatant from each tube the precipitate was resuspended in 1 ml water and absorbance of each was monitored at 280 nm by spectrophotometer to allow for estimation of the amount of antibody in supernatant and precipitated complex. The result suggests that the recovery of the antibody in the precipitate increases with carboxyl group concentration (substitution×polymer concentration). Even at these small volumes, where one expects to lose antibody to nonspecific tube wall adsorption and other phenomena, antibody recovery of >80% was obtained with 20% CMD 40000 and carboxyl density of 3.24 mmol/g. Up to 82% antibody was found in the precipitate, and samples could be readily re-dissolved even in distilled water.

TABLE 7
Precipitation and recovery of Ab using 20% CMD of varied MW and ligand density.

| CMD | Carboxyl | Carboxyl | mg Ab | mg Ab | mg Ab | % Ab in | % Ab in | Total |
|---|---|---|---|---|---|---|---|---|
| MW | (mmol/g) | (M) | added* | Supernat. | ppt. | Supernat. | ppt | recovery |
| 10000 | CMD L-0.53 | 0.33 | 6.1 | 2.45 | 3.4 | 40 | 56 | 96 |
| | (1.65) | | | | | | | |
| 40000 | CMD 0.93 | 0.45 | 6.1 | 0.65 | 4.22 | 10 | 69 | 79 |
| | (2.26) | | | | | | | |
| 10000 | CMD L-0.91 | 0.53 | 6.1 | 1.55 | 4.06 | 25 | 66 | 91 |
| | (2.66) | | | | | | | |
| 40000 | CMD 1.39 | 0.64 | 6.1 | 0.22 | 5.03 | 3.6 | 82 | 86 |
| | (3.24) | | | | | | | |

*Added to 1.2 ml sample thus 6.1 mg is approx. 5.1 mg/ml.

### Example 6

### Precipitation of mAb from Different Crude Feeds with PAA and CMD

Different monoclonal mAb fermentation feeds, termed P4, P5 and 51 produced in Chinese Hampster Ovary (CHO) cell fermentations were clarified of cells and cell debris in normal manner using centrifugation. Sample type 51 was produced in a disposable WAVE™ Bioreactor (GE Healthcare, Uppsala), thereafter sample type 51 is also called WAVE 51. A control sample of 51 feed was clarified in a Breox 50 A 1000 thermoresponsive polymer containing aqueous polymer two phase system at 40° C. (i.e. above polymer Tc). See Methods and Materials above for more information. Additional information on phase systems formed with Breox and other “EOPO” polymers such as Tergitol and Ucon is available from various sources e.g. H. O. Johansson et al, 1998 (above). Samples of the various feeds which had been clarified by centrifugal or aqueous polymer two phase partition were subjected to precipitation with different concentrations of PAA and CMD polymers, using different buffer and NaCl concentrations (see Table 8). After flocculation, centrifuge aided precipitation and removal of the supernatant from each tube the precipitate was resuspended in water and analyzed for mAb content by protein A chromatographic analysis (described above under Methods).

The results obtained from these experiments showed that:
No precipitation of mAb occurs when buffer and NaCl were excluded from the system (experiment 2).
No precipitation of mAb occurs when the concentration of PAA was reduced from 10% to 3% even when relatively high buffer NaP and NaCl salt concentrations were used (experiments 3 and 3a).
mAb precipitate recoveries of 78-88% were achieved with 10% PAA and 20% CMD systems using high buffer and NaCl concentrations, even when high concentration of mAb feeds (Wave 51 and Wave 51 ATPS) were used (experiments 4 and 1d-1e). However, when different feeds (P4 and P5) with relatively low mAb concentrations were used, mAb recovery in the precipitate decreased to 56-61% (experiments 1 and 1b) although the latter may simply be due to loss of antibody sample in analysis due to the high concentration effect of the co-precipitation yielding a sample whose volume was <<1% of the starting solution.

TABLE 8
Precipitation and recovery of mAb from different crude feeds using PAA 15000 and
CMD 40000 (1.39) and different buffer/salt conditions

| | | | | | | | mg | | | | |
|---|---|---|---|---|---|---|---|---|---|---|---|
| | | | | | ml | mg | mAb | mg | % mAb | % mAb | Total |
| | NaP/NaCl | Vol. | Polymer | mAb | mAb | mAb | in | mAb in | in | in | recovery |
| No | (mM) | (ml) | (% w/w) | feed | added | added | ppt. | Supern. | ppt. | Supern. | % |
| 1 | 200/150 | 5 | 10% PAA | P5 | 2.18 | 0.33 | 0.19 | 0.12 | 56 | 35 | 91 |
| 2 | 0/0 | 5 | 10% PAA | P5 | 2.18 | 0.33 | 0 | 0.32 | 0 | 95 | 95 |
| 3 | 200/150 | 5 | 3% PAA | P5 | 2.18 | 0.33 | 0 | 0.3 | 0 | 91 | 91 |
| 3a | 200/150 | 5 | 3% PAA | P5 | 2.18 | 0.33 | 0 | 0.31 | 0 | 94 | 94 |
| 4 | 200/150 | 1.25 | 20% CMD | Wave 51 | 0.7 | 0.81 | 0.68 | 0.15 | 85 | 18 | 103 |
| 1b | 200/150 | 5 | 10% PAA | P4 | 2.18 | 0.83 | 0.50 | 0.23 | 61 | 28 | 89 |
| 1d | 200/150 | 5 | 10% PAA | Wave 51 | 2.18 | 2.51 | 2.21 | 0.27 | 88 | 11 | 99 |
| 1e | 200/150 | 5 | 10% PAA | Wave 51 | 2.18 | 1.80 | 1.40 | 0.25 | 78 | 14 | 92 |
| | | | | APTP | | | | | | | |

PAA refers to NaPAA 15000, CMD refers to CMD 40000 (1.39 substitution). APTP refers to feed clarification in a Breox EOPO polymer containing aqueous polymer two-phase system.

The host cell protein (HCP) content was analyzed by an enzyme linked immunoassay using a Gyrolab system (Gyros, Uppsala) (Table 9). Results show a HCP reduction of 88-94% in the precipitated mAb samples and that most of the HCP remained in the supernatants. They also indicate that the precipitation method interfaces well with antibody samples not just in clarified feed but also those from aqueous polymer phase system phases such as the protein-rich upper phase of thermoseparated Breox EOPO polymer containing two phase system. In this regard residual EOPO polymer in the upper phase did not appear to interfere with antibody precipitation. A result which is not unexpected given the uncharged nature of the Breox polymer.

TABLE 9
HCP data for systems with PAA 15000 or CMD 40000 (1.39).

| | | | Total ng HCP | | | | |
|---|---|---|---|---|---|---|---|
| Exp. | Total mg | Total ng HCP | recovered in | Total ng HCP | Total HCP | % HCP | Ppm |
| No | mAb in ppn | added (feed) | ppt. | in supern. | recovery % | reduce | HCP |
| 4 | 0.681 | 23100 | 2700 | 13750 | 71 | 88 | 3964 |
| 1d | 2.21 | 71940 | 6350 | 55000 | 85 | 91 | 2873 |
| 1e | 1.80 | 47960 | 3000 | 33250 | 76 | 94 | 1660 |

### Example 7

### Precipitation of mAb With CMD or PAA at 10 ml Scale

To investigate accuracy and reproducibility of the method, some of the experiments presented in Table 8 were repeated at 10 ml scale. The results presented in Table 10 indicate that high level mAb recoveries in the precipitates (76 and 99%) were achieved with 10% PAA or 20% CMD 4000 systems, under conditions of relatively high mAb concentration (i.e., Wave 51 APTP clarified feed) and conductivity. Table 11 shows HCP reduction of >94% in the precipitated mAbs with most of the HCP remaining in the supernatants. These results are similar to results obtained above at 1.2 to 5 ml scale and suggest both the scalability and robustness of the method, as well as its ability to be screened using low volume test systems such as microtitre plates or small volume test tubes.

TABLE 10
Precipitation and recovery of mAb sample from a crude feed clarified by APTP
partition using NaPAA 15000 or CMD 40000 at 10 ml scale.

| | | | ml | mg | | | % mAb | % mAb | Total |
|---|---|---|---|---|---|---|---|---|---|
| exp | Polymer | | mAb | mAb | mg mAb | mg mAb in | in | in | recovery |
| No | % | mAb | add | added | in ppn | supernatant | ppn | Supernatant | % |
| 4 | 20% | Wave | 4.5 | 3.84 | 3.82 | 0.02 | 99 | <1% 0 | 99 |
| | CMD | 51 | | | | | | | |
| | | APTP | | | | | | | |
| 1e | 10% | Wave | 5.0 | 4.27 | 3.24 | 0.89 | 76 | 21 | 97 |
| | PAA | 51 | | | | | | | |
| | | APTP | | | | | | | |

*10% NaPAA 15000 or 20% CMD 40000 (1.39) and 150 mM NaCl, 200 mM NaP pH 7.0

TABLE 11
Host Cell Protein (HCP) data related to experiments in Table 10.

| | | | | Total mg | % Total | | |
|---|---|---|---|---|---|---|---|
| | Total mg | Total mg HCP | Total mg | HCP in | HCP | % HCP | HCP |
| Exp./Feed | mAb in ppn | added (feed) | HCP in ppt. | supern. | recovery | reduced | (ppm) |
| 4/Wave 51 | 3.82 | 108 | 7.0 | 91 | 90 | 94 | 1832 |
| APTP* | | | | | | | |
| 1e/Wave 51 | 3.24 | 120 | 5.5 | 115 | 100 | 95 | 1700 |
| APTP | | | | | | | |

*Aqueous polymer two phase (APTP) system decribed in Methods above.

### Example 8

### mAb Precipitation with PAA at 200 ml Scale

In order to further test scalability and reproducibility, a precipitation system based on 10% NaPAA 15000, 150 mM NaCl, 200 mM NaP pH 7, and mAb 51 feed from Wave Bioreactor, clarified by thermoresponsive APTP system partition (see above) was processed at 200 ml scale in duplicate (Table 12). After complex formation and precipitation, plus removal of the supernatant, each precipitate was resuspended in 50 ml of water. In this case the resuspension volume was high so that further analyses could be run, and also some samples stored frozen for future analyses. Samples of resuspended solutions and supernatants were analyzed for mAb content by adjusting salt concentration and pH so that the antibody would bind to a Mabselect Sure protein A affinity column (which allowed for analysis of the amount of mAb present). Protein recoveries for the supernatants and precipitate are presented in Table 12. As expected approximate mAb recoveries of >86% were achieved in the complexes. Furthermore the remaining mAb appeared to be in the supernatant where, for example it might be readily subjected to a second precipitation step so as to increase the amount of purified mAb. Observe that the second precipitation step could be accomplished simply by adding a small amount of NaPAA to the supernatant so as to raise the NaPAA level back to 10%. In this example approximately 86% of mAb was found in the precipitate and 18% in the supernatant (total is 104% due to standard errors). If we assume the worse case and that 82% of mAb was in the precipitate (K=82/18=4.6) and the second precipitation yielded a similar ratio then pooling the precipitates from the first and second step would result in approx. 97% of mAb in complexed form.

Note also that in the above example the Protein A based MabSelect Sure affinity column was run in normal manner and gave normal looking chromatograms (not shown). This suggested that it is possible to include a precipitation or partition followed by precipitation step upstream (previous) to a protein A type of affinity capture step. Such a step might involve capture filtration, or capture chromatography using packed or expanded particle beds or monolithic columns.

Residual, neutral APTP system related polymers may not negatively affect (i.e. may have little influence or even a positive influence) on follow on affinity or ion exchange chromatography (US 20070213513 A1). As regards residual PAA in the chromatogaphed mAb sample (which is typically only a small percentage of the PAA in the initial precipitation solution) it should be noted that PAA has a net negative charge as does the protein A column as the pl of protein A analogues are approximately 5. That, together with its relatively small MW should allow for the PAA to pass a protein A column (or other negatively charged column such as a cation exchange column) in the flow through. The lower MW of the polymer may also afford its removal by a specific filtration step, or simply by nonspecific adsorption during other normal processing steps.

TABLE 12
200 ml Scale Precipitation and recovery of mAb from
(APTP system clarified) crude feed using10% PAA 15000
and 200 mM NaP buffer and 150 mM NaCl.

| | ml | mg | mg | mg mAb | | % mAb | |
|---|---|---|---|---|---|---|---|
| Exp. | mAb | mAb | mAb in | in | % mAb | in | % Total |
| No. | added | added | ppt. | Supern. | in ppn | Supern. | recovery* |
| 1 | 100 | 84.6 | 73.4 | 15.4 | 87 | 18 | 105 |
| 2 | 100 | 84.6 | 73 | 15 | 86 | 18 | 104 |

*It is assumed that these values reflect errors of ≦10%.

### Example 9

### Chromatography of Re-Dissolved Precipitation on a Capto MMC Column

This experiment was conducted in order to verify that resuspended mAb precipitate sample could be processed on other media including cation exchange media and mixed mode media. Capto™ MMC is a multimodal cation exchange media commercially available for bioprocessing (GE Healthcare, Uppsala). Information on its structure and use is available through the supplier either directly by mail or via website (i.e. Optimizing elution conditions on Capto MMC using Design of Experiments, GE Healthcare publication 11-0035-48. Capto MMC Data File, GE Healthcare publication 11-0025-76). It has been designed for use at normal to high flow rates (at least 600 cm/h in large columns) and normal to relatively high mobile phase salt concentrations (e.g. 5 to 50 mS/cm) and would appear ideal for processing precipitate samples resuspended in minimal volume solutions which contain high concentrations of target proteins (in net positive state) plus residual salt and negatively charged polymers.

Precipitate was produced using a sample of real feed from Chinese Hampster Ovary (CHO) cell fermentation feed (mAb 5 g/L) which had been fermented in disposable Wave Bioreactor and then clarified by aqueous polymer two phase (APTP) partitioning in the same disposable Wave Bioreactor. Precipitation was induced by modification of mAb containing phase (which held over 90% of initial mAb subjected to APTP) by adding polymer and salts to achieve 10% (w/w) NaPAA 15000, 200 mM NaP pH 7 and 150 mM NaCl. Precipitate, which contained approximately 90% of mAb from partitioning, was estimated as <2% (v/v) of total precipitation system solution volume. It was dissolved in 10 volumes of buffer consisting of 250 mM NaCl and 50 mM NaAcetate adjusted to pH 5.5 with acetic acid. Approximately 17 ml sample was applied to Capto MMC column at 0.5 mL per min (2 min residence time) on 1 ml HiTrap column packed with Capto MMC. Elution (Buffer B) was at 100 mM NaP pH 7.6 containing 1M NaCl.

The goal was to bind mAb on the column and allow most of the remaining HCP to flow through. The bound mAb would then be eluted from the column by increasing pH and salt concentration. Any residual protein, including possibly some contaminants would then be eluted at the highest pH and salt concentration. FIG. 8 shows chromatography data of resuspended precipitate on Capto MMC column. Fractions from the chromatography experiment were collected and analyzed for HCP and DNA content, and the data is compared with that of crude feeds and the supernatant and the precipitate (Table 13). For purity check a sodium dodecyl sulphate, polyacrylamide gel electrophoresis (SDS PAGE) analysis was also performed (FIG. 9).

The chromatogram in FIG. 8 shows a broad initial peak in the eluate which suggests that a significant amount of mAb flowed through the column (i.e. the initial adsorption buffer concentration or amount of mAb was too high for the small column used). SDS PAGE confirmed this (FIG. 9). However much (50% or more) mAb appeared to be captured by the column as evidenced by sharp peak in the middle of the chromatogram run (see Table 13 and FIGS. 8 and 9). The negatively charged PAA polymer was believed to be eluted with negatively charged HCP and perhaps toxins, virus and other negatively charged contaminants (not analysed) in the flow through. Adsorbed contaminants would be (partially) removed in a high pH, high conductivity washing step.

Table 13 shows a reduction of HCP in the eluate (fraction A6) to about 800 ppm. Significant reduction of DNA was archived as indicated by the level of DNA content which is below the detection limit of the assay employed.

TABLE 13
HCP & DNA data from re-dissolved precipitate,
supernatants and chromatography
on Capto MMC, 200 ml scale.

| | mg | % mAb | Total ng | ppm | total ng | ppm |
|---|---|---|---|---|---|---|
| Sample | mAb | recovery | DNA | DNA | HCP | HCP |
| Feed Wave | | | 781522 | 35203 | 731000 | 32927 |
| 51 APTP | | | | | | |
| supernatant | | | | | 605200 | 27261 |
| Resuspended | 22.2 | | 5244 | 236 | 34850 | 1570 |
| in pH 5.5 | | | | | | |
| pool A1-A4 | 7.6 | 34 | 5148 | 677 | 8700 | 1144 |
| Fraction A6 | 11.03 | 50 | | Not | 9000 | 815 |
| | | | | Detectable | | |

### Example 10

### Precipitation of Antibody Fragment (Fab)

Antibody fragments (Fabs) typically have a MW one third that of parent mAb but often exhibit ion exchange chromatography behavior similar to parent mAbs and similar titration curves (in relation to MW and number of residues, e.g. For mAbs: C. Harinarayan, J. Mueller, A. Ljunglof, R. Fahrner, J. Van Alstine, R. van Reis, An Exclusion Mechanism in Ion Exchange Chromatography, Biotechnology and Bioengineering 95 (2006) 775-787; and For Fabs: A. Ljunglof, K. M. Lacki, J. Mueller, C. Harinarayan, R. van Reis, R. Fahrner, J. M. Van Alstine, Ion Exchange Chromatography of Antibody Fragments, Biotechnology and Bioengineering 96 (2007) 515-524). The similar properties such as relative hydrophobicity, charge density, pl of Fabs suggests they should be able to be complexed by the methods noted above. However smaller size of Fabs, with reduced surface area for polymer interaction plus greater diffusivity suggests they may complex to a lesser degree than mAbs under some conditions. To test this, an internally supplied Fab solution at 3 g/L was combined with polymer and salt to form a 5 ml system with 0.6 g/L Fab in 10% (w/w) NaPAA 15000, 200 mM NaPhosphate pH 7, 150 mM NaCl. A visible precipitate formed. UV analysis (A280 nm) suggested that 20% of the Fab was complexed under these conditions. Further experimentation was not performed but it should be readily possible to significantly increase this promising result via methods noted above including an increase in polymer or salt concentration, increase in polymer MW, altering pH, temperature, etc.

It is interesting to note that in addition to Abs and related proteins some other proteins may be amenable to processing via this approach. The proteins should be net positive (under the pH conditions used) and if possible offer some degree of surface hydrophobicity. As noted above protein size will also play a role. Examples include several proteins of commercial interest such as serum albumins and chymotrypsins as well as insulins and several other hormones. Thus Alves et al. showed that in PEG and salt plus PEG and dextran systems insulin tends to favor the upper phase with partition coefficients as high as 10 (Jose G. L. F. Alves, Lucy D. A. Chumpitaz, Luiza H. M. da Silva, Telma T. Franco, and Antonio J. A. Meirelles: Partitioning of whey proteins, bovine serum albumin and porcine insulin in aqueous two-phase systems, Journal of Chromatography B, 743 (2000) 235-239). Fuentes et al.

recently studied the interaction of various proteins with CMD polymers noncovalently adsorbed on chromatographic substrates. They noted that E coli HCP were bound to the CMD modified matrix at pH 5 but not at pH 7, that reduction in HCP interaction with the polymer coated surfaces decreased as ionic strength increased, and that at pH 7 the relatively hydrophobic basic protein chymotrypsin (pl approx. 9) was not released from the polymer until >200 mM NaCl had been added to the mobile phase. They also noted that polymer interaction appeared to confer some structural stability on the proteins which retained their native activity (Manuel Fuentes, Benevides C. C. Pessela, Jorgette V. Maquiese, Claudia Ortiz, Rosa L. Segura, Jose M. Palomo, Olga Abian, Rodrigo Torres, Cesar Mateo, Roberto Fernandez-Lafuente, and J. M. Guisan, Reversible and Strong Immobilization of Proteins by Ionic Exchange on Supports Coated with Sulfate-Dextran, Biotechnol. Prog. 20 (2004) 1134-1139).

All patents, patent publications, and other published references mentioned herein are hereby incorporated by reference in their entireties as if each had been individually and specifically incorporated by reference herein. While preferred illustrative embodiments of the present invention are described, one skilled in the art will appreciate that the present invention can be practiced by other than the described embodiments, which are presented for purposes of illustration only and not by way of limitation. The present invention is limited only by the claims that follow.

1. A method for isolating a biomolecule, which method comprising the steps of:
(a) providing an aqueous sample containing said biomolecule;
(b) mixing the aqueous sample with a negatively charged polymer in the presence of a salt, under conditions such that said polymer selectively complexes and flocculate the biomolecule to form a mixture of precipitate including the biomolecule;
(c) separating the biomolecule precipitate from the aqueous liquid; and
(d) resuspending the biomolecule in a resuspension buffer.

2. The method of claim 1, wherein the biomolecule is a protein including a hormone or a polyclonal antibody or a monoclonal antibody or antibody derived protein.

3. The method of claim 1, wherein the antibody is an IgG antibody.

4. The method of claim 1 wherein the biomolecule is an antibody fragment (Fab).

5. The method of claim 1, wherein the negatively charged polymer is a polyacrylic acid (PAA).

6. The method of claim 5, wherein the PAA has a molecular weight of greater than 5 kD and a concentration of greater than 3% (w/v).

7. The method of claim 1, wherein the polymer is carboxymethyl-Dextran (CMD) or other carboxy modified polymer, or other polyacid or other biodegrading polyacid polymers.

8. The method of claim 1, wherein said salt is selected from NaPhosphate, NaCl, NaCitrate and NaSulfate.

9. The method of claim 8, wherein concentration of said salt is greater than 50 mM.

10. The method of claim 1 wherein the pH of the mixture in step (b) is between about 5 and about 9.

11. The method of claim 1, wherein the pH of the mixture in step (b) is around pH 7.

12. The method of claim 1, wherein the aqueous sample is selected from the group consisting of clarified fermentation product from a prokaryotic or eukaryotic expression system, viral culture systems, whole blood, clarified blood, recombinant milk, recombinant plant solutions, and any other aqueous sample containing the biomolecules of interest.

13. The method of claim 12, wherein clarification, or other earlier sample purification separation step is performed by centrifugation or partitioning in one or more aqueous multiphase separation systems including those formed by one or two water soluble polymers in the presence of various buffers or salts.

14. The method of claim 1, wherein the separating comprises:
(a) centrifuging the mixture to form the precipitate and the aqueous liquid; and
(b) removing the aqueous liquid from the precipitate.

15. The method of claim 1, wherein the separating comprises filtering the mixture to isolate the complex from the aqueous fluid.

16. The method of claim 1, wherein said precipitated biomolecules is resuspended in an aqueous buffer having a pH between 3 and 9, or water.

17. The method of claim 5, wherein residual PAA or other polyacid in the precipitate is removed by scavenging after step (d).

18. The method of claim 5, wherein residual PAA or other polyacid in the precipitate is removed, after step (d), by an aqueous multiphase system.

19. The method of claim 5, wherein residual PAA or other polyacid in the precipitate is removed, after step (d), by allowing it to flow through a chromatographic or filtration or other (monolithic) capture media, which significantly adsorbs said biomolecule but not the PAA or other polyacid.

20. The method of claim 5, wherein residual PAA or other polyacid in the precipitate is removed, after step (d), by allowing it to flow through a chromatographic or filtration or other (monolithic) size exclusion media where PAA or other polyacid has different rate of flow or degree of hindrance than said biomolecule.

21. The method of claim 1, further comprising one or more additional purification steps which may include further aqueous phase partition or precipitation steps.

22. The method of claim 21, wherein the additional purification steps include chromatography using a multimodal cation exchanger; Protein A affinity column, hydrophobic interaction column and cation exchange.

23-27. (canceled)
